(19) United States
(12) Patent Application Publication
(10) Pub. No.: US 2015/0069263 A1
(43) Pub. Date: Mar. 12, 2015

(54) Light Emitting Disconnectable Blow-Dry Brush

(71) Applicant: Daniel MOYAL, (US)

(72) Inventor: Daniel Moyal, Tel Aviv (IL)

(21) Appl. No.: 14/382,773

(22) PCT Filed: Feb. 14, 2013
(86) PCT No.: PCT/IB2013/051184

§ 371 Date: Sep. 4, 2014

Related U.S. Application Data
(60) Provisional application No. 61/608,123, filed on Mar. 8, 2012.

(51) Int. Cl. A45D 20/52; A61L 2/10; A46B 9/02; A46B 13/00; A46B 15/00; A46B 17/06

(52) U.S. Cl. CPC A45D 20/52; A46B 15/0034; A46B 17/065; A46B 9/023; A46B 13/001; A46B 15/0051; A46B 15/0036; A61L 2/10
USPC 250/455.11; 15/105; 15/23; 132/271

(57) ABSTRACT

A round hair-brush comprising: a hollow body comprising holes in its circumference; and a handle connected with the hollow body on one end thereof, wherein the body comprises: at least one electric generator having a shaft; at least one LED electrically connected with the at least one electric generator; and at least one propeller electrically connected with the at least one electric generator shaft, the at least one propeller configured to turn upon receiving air current from a hair-dryer and transfer the rotation power to the generator, the generator configured to transform the rotation power to electric power thereby lighting up the at least one LED.

### CROSS-REFERENCE TO RELATED PATENT APPLICATIONS

This patent application claims priority from and is related to U.S. Provisional Patent Application Ser. No. 61/608,123, filed 8 Mar. 2012, this U.S. Provisional Patent Application incorporated by reference in its entirety herein.

### FIELD OF THE INVENTION

The present invention relates to hair grooming appliances and more particularly to blow-dry round hair brush having a light source.

### BACKGROUND

Blow-drying is a common procedure performed by hairdressers, using a blow dryer and a round hair brush, such as hair brush 100 depicted in FIG. 1. Beginning at the roots, the hairdresser gently pulls the brush through the hair to the ends while blow-drying it.

The brush that comes in contact with many clients' heads must be sterilized between uses.

Various hair-brush sterilizers are in use, such as Hair Salon UV Tool Sterilizer H6501, available from Xinhui Mingzhuo Salon & Beauty Parlor Ambience Co., Ltd., China.

These sterilizers are stand-alone receptacles into which the brushes are inserted and which sterilize the brushes using UV light or other sterilizing agents.

While in the sterilizer, the brush is not available for use by the hairdresser, which sometimes leads to less meticulous use of the sterilizer, namely the brush is not being sterilized following each use session.

### BRIEF DESCRIPTION OF THE DRAWINGS

For better understanding of the invention and to show how the same may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 depicts a round hair-brush commonly used for blow-drying hair;

FIG. 2 is a schematic representation of an embodiment of the device according to the present invention;

FIG. 3 is a schematic representation of another embodiment of the device according to the present invention;

FIG. 4 is a schematic representation of another embodiment of the device according to the present invention;

FIG. 5 is a schematic representation of yet another embodiment of the device according to the present invention;

FIG. 6 is a schematic representation of yet another embodiment of the device according to the present invention;

FIG. 7 is a schematic representation of yet another embodiment of the device according to the present invention;

FIGS. 8A through 8E show a schematic representation of yet another embodiment of the device according to the present invention; and

FIG. 9 is a schematic drawing of a standalone hairbrush sterilizer according to an embodiment of the present invention.

### DETAILED DESCRIPTION OF PREFERRED EMBODIMENTS

The present invention provides new devices and methods for sterilizing round hollow hair brushes. The devices and methods may have additional uses, as will be apparent from the description below.

The device according to the present invention is a light generating system, driven by the blow-dryer's wind directed at the brush or operated manually.

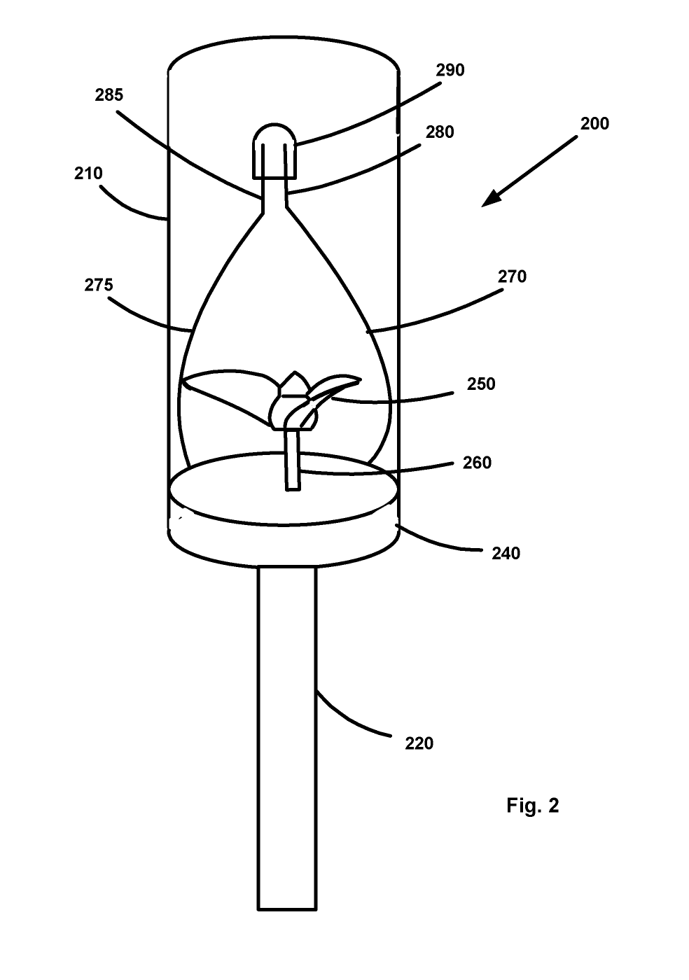

FIG. 2 is a schematic representation of an embodiment of the device according to the present invention.

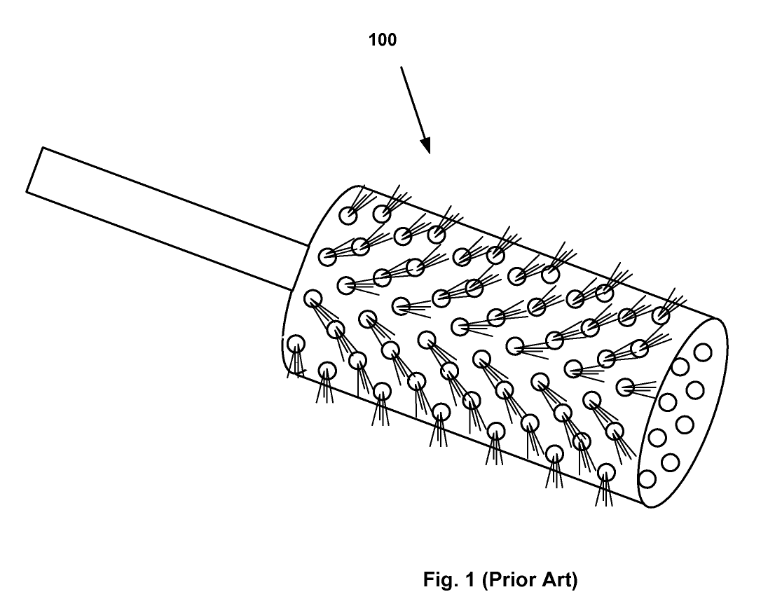

A round brush 200 comprises a hollow body 210 and a handle 220. The holes and bristles on the circumference of the brush body (as shown in FIG. 1) are not shown for clarity. According to the embodiment of FIG. 2, an electric generator e.g. dynamo generator 240 is attached to the end of the hollow body 210 near the handle 220. Attaching the generator may be done for example by screwing or by gluing or by any attachment method known in the art. The generator may be any electric generator known in the art and capable of generating a current of up to 500 ma, such as for example, a small power generator, provided from Dongguan Yingyi Hardware Electric Technology Co., Ltd., China, or Hand Dynamo, provided from Shenzhen China Trader Strength Electronics Co., Ltd., China.

Two electric wires (270, 275) emerging from the generator 240 are connected respectively at their other ends to the anode 280 and cathode 285 pins of a LED (Light emitting Diode) 290. Electric wires (270, 275) and all the electric wires described in the following embodiments are preferably heat resistant wires, such as available from Thermal Wire & Cable Corp., www.thermalwire.com.

The LED may be any LED having a current consumption of 20 to 150 ma, depending on its size and color. The LED 290 may be, for example, 5 mm ultra violet blue led diode, available from Hubei Kento Electronic Co., Ltd., China, or 5 mm led, available from Tyki lighting technology co., ltd, China, or Infrared LED, available from Zhongshan Liguang Electronic Co., Ltd., China.

The LED color may be selected according to the function to be performed. For example, a UV LED with wavelength 240-260 nm may be used for sterilization purposes, an IR LED may be used for therapeutic purposes such as skin therapy and any other LED spectrum may be used for visually enhancing the blow-drying experience.

A propeller 250 is rotatably mounted on a shaft 260, which is in turn connected to the generator 240 shaft (not shown).

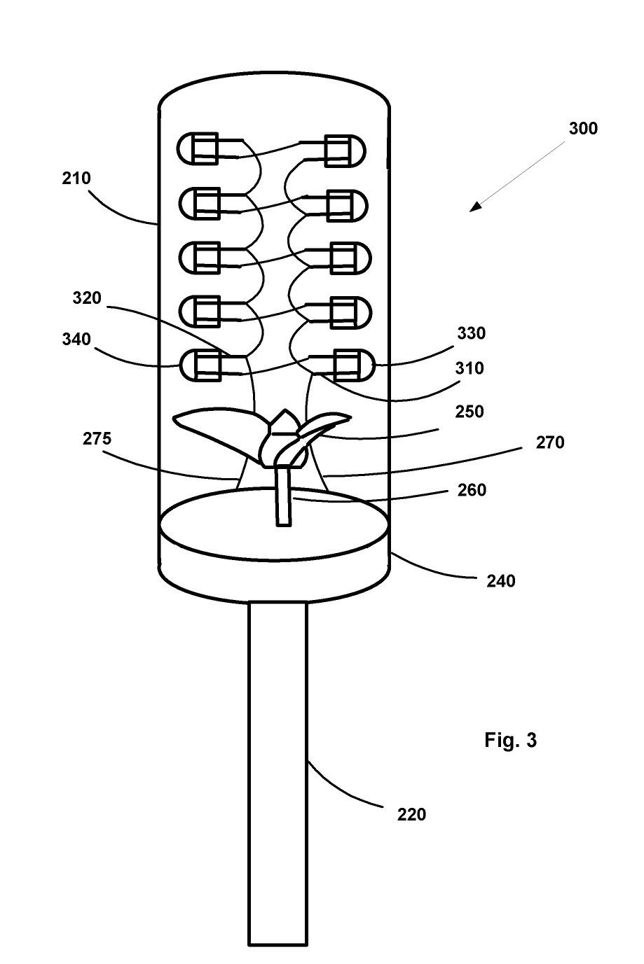

FIG. 3 is a schematic representation of another embodiment of the device according to the present invention.

A round brush 300 comprises a hollow body 210 and a handle 220. The holes and bristles on the circumference of the brush body (as shown in FIG. 1) are not shown. According to the embodiment of FIG. 3, an electric generator, such as a dynamo generator 240, same as in the embodiment of FIG. 2, is attached to the end of the hollow body 210 near the handle 220.

Two electric wires (270, 275) emerging from the generator 240 are connected respectively at their other ends to the anode 310 and cathode 320 pins of two LEDs 330, 340. LEDs 330 and 340 may be further connected to a chain of multiple LEDs, depending on the LEDs size and the brush size,

A propeller 250 is rotatably mounted on a shaft 260. The propeller shaft 260 is rotatably connected to the generator shaft (not shown).

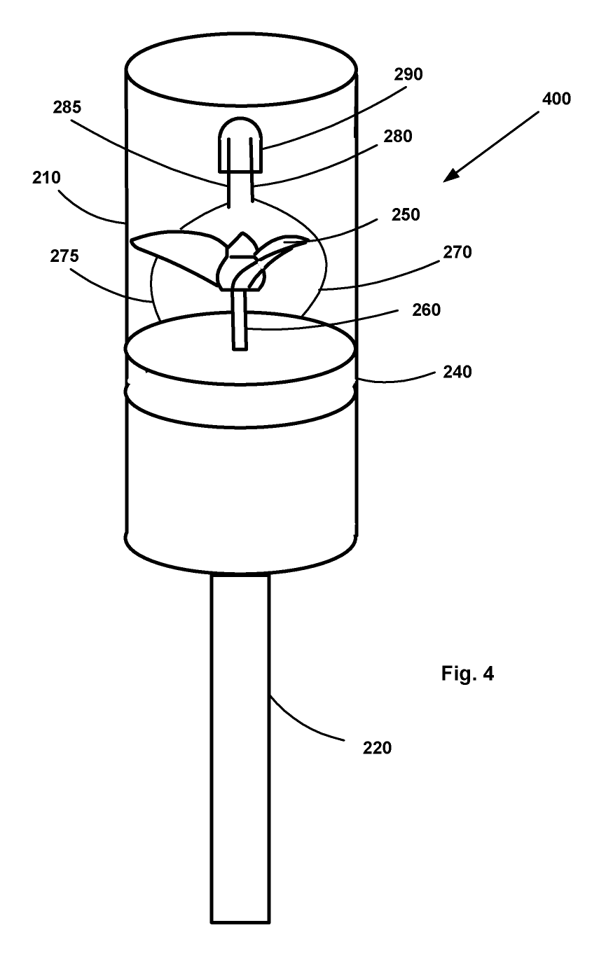

FIG. 4 is a schematic representation of another embodiment of the device according to the present invention. The device of FIG. 4 is similar to the device of FIG. 2, except that the generator and propeller may be installed anywhere along the cylindrical body 210 of the brush.

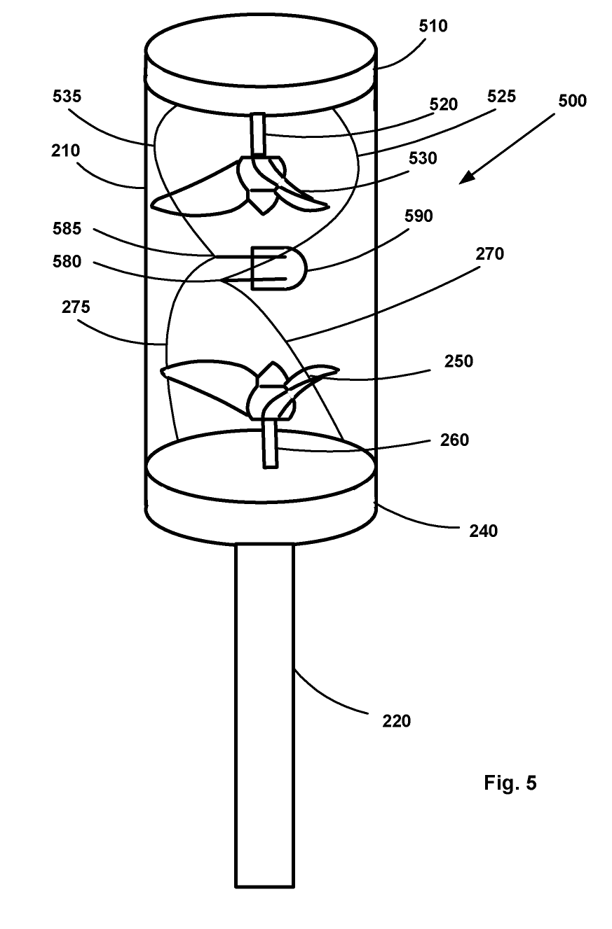

FIG. 5 is a schematic representation of another embodiment of the device according to the present invention. The device of FIG. 5 comprises two electric generators (240, 510) attached respectively on both ends of hollow body 210. Each electric generator is respectively connected to a propeller (250, 530) through a shaft (260, 520). Two electric wires (270, 275) emerging from electric generator 240 and two electric wires (525, 535) emerging from electric generator 510 are connected respectively at their other ends to the anode 580 and cathode 585 pins of a LED 590.

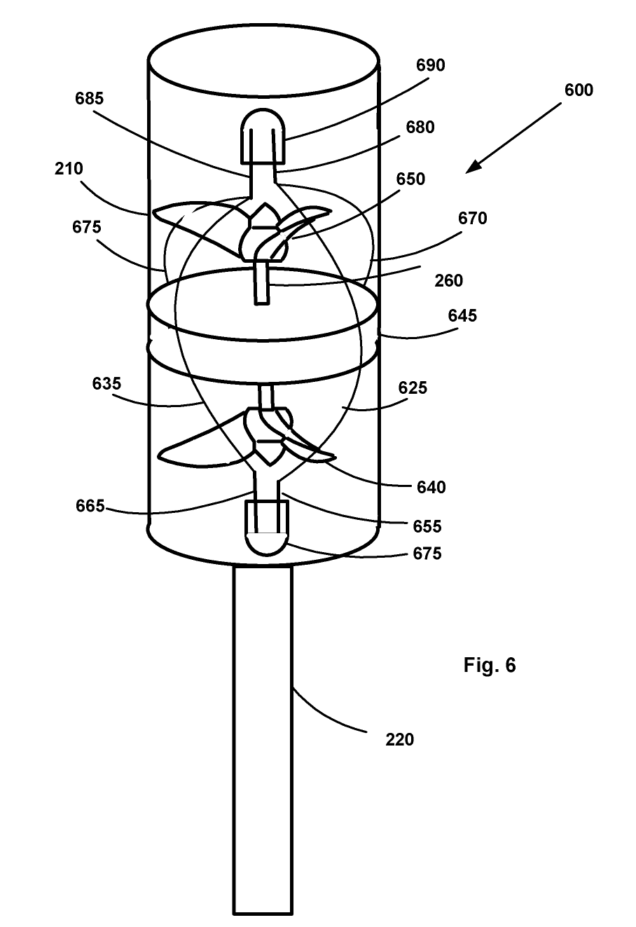

FIG. 6 is a schematic representation of another embodiment of the device according to the present invention. The device of FIG. 6 comprises a single electric generator 645, mounted substantially in the middle of hollow body 210. The electric generator 645 is connected to two propellers (650, 640) on both sides thereof. Two electric wires (670, 675) emerging from the electric generator 645 are connected respectively at their other ends to the anode 680 and cathode 685 pins of a LED 690, which are respectively connected to anode 655 and cathode 665 pins of a second LED 675.

In operation, the hairdresser performing blow-drying pulls the brush through the hair and aims the blow-dryer at the brush. The air current from the blow-dryer impinges on the propeller(s) blades, causing them to rotate. The rotation is transferred to the respective electric generator(s) which transform the mechanical power into electric power, which is transferred by the generator(s) to the LED/LEDs, causing them to lighten up.

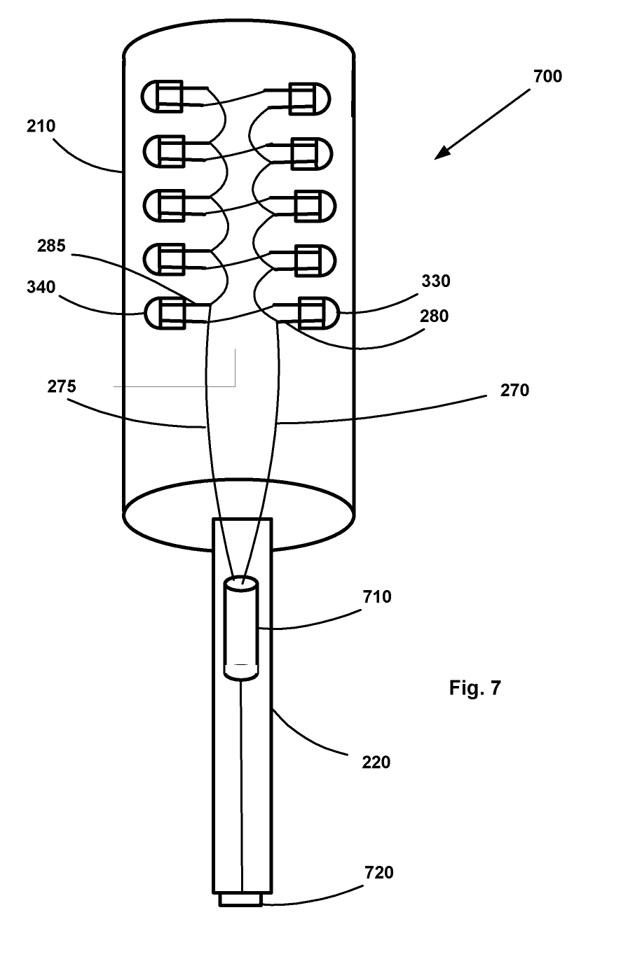

FIG. 7 is a schematic representation of another embodiment of the device according to the present invention. The device of FIG. 7 is similar to the device of FIG. 3, except that the power driving the LEDs is not generated by a propeller-driven electric generator, but rather by a battery 710 mounted in the hair brush handle 220 and operated by a switch 720. The switch 720 may be installed on the far end of handle 220, as depicted in FIG. 7, or at any other suitable location. The battery 710 may be a rechargeable battery.

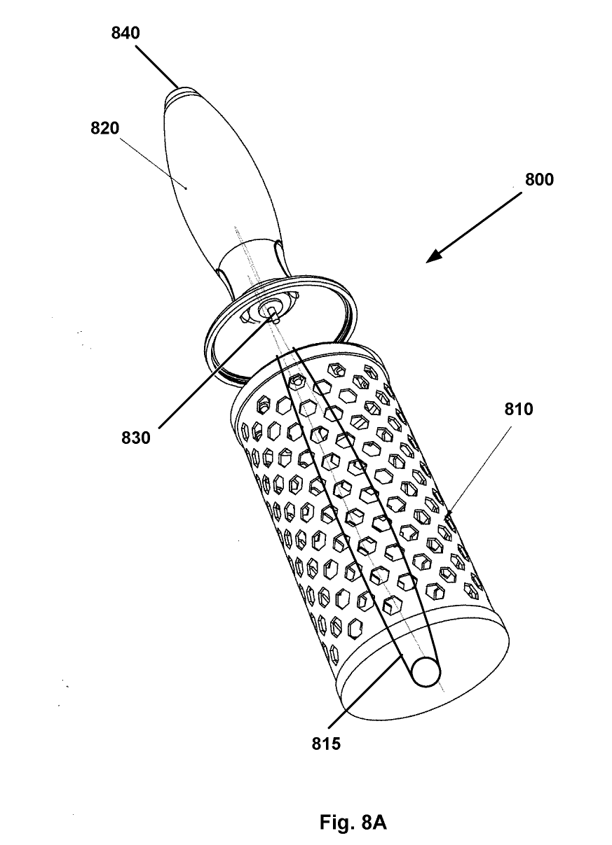

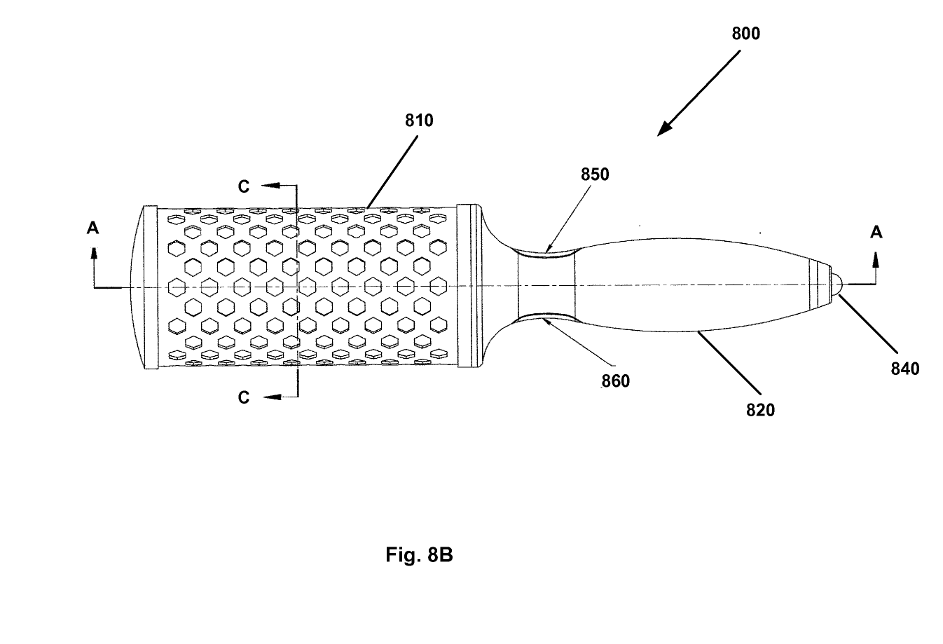

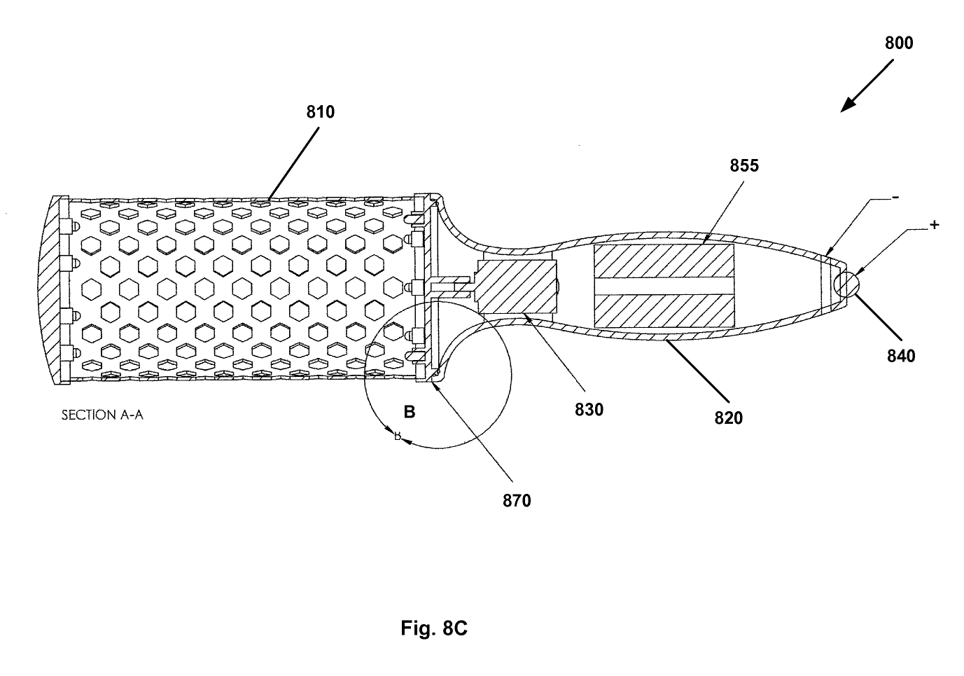

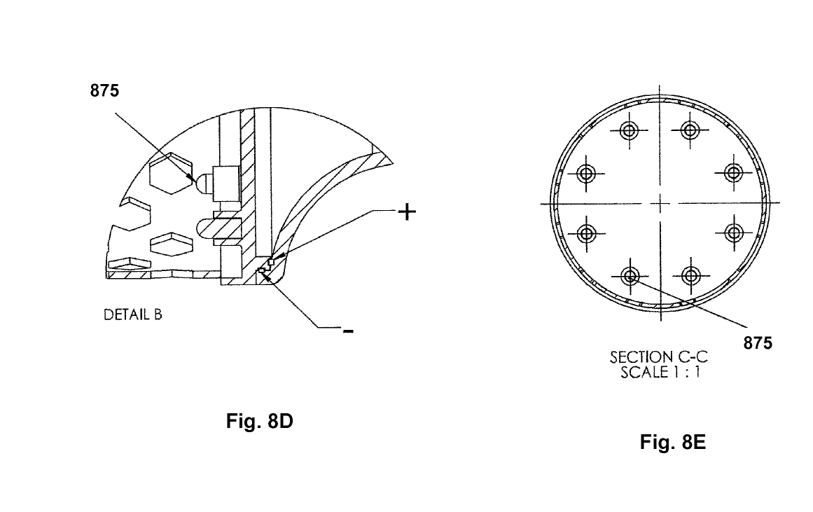

FIGS. 8A through 8E show a schematic representation of yet another embodiment of the device according to the present invention.

The device 800 comprises a round brush body 810 and a handle 820. The handle 820 comprises a motor 830, rotatably connected to a central shaft in the body 810 (not shown) and a battery 855 for supplying voltage to the motor 830 and to the LED/LEDs 875, as explained above in conjunction with FIG. 7 and as shown in FIG. 8D. The battery 855 may be a rechargeable battery and charging contacts 840 may be mounted on the distal end of the handle 820. The central shaft of the brush may be connected with a planetary gear (not shown) which reduces the motor speed and provides a greater moment to the brush body.

A LED/LEDs On/Off button 850 may be mounted on the handle 820.

An On/Off button 860 may be mounted on the handle 820, for controlling the motor 830 operation. With each push of the On/Off button 860 the motor performs an up to 360 degrees rotation, left or right, where the rotation degree and direction is predefined, causing the brush body 810 to rotate accordingly. The rotation is controlled by an electronic circuit. In a preferred embodiment, each rotation is predefined to 180 degrees, to best simulate a hairdresser's manual rotation of the brush.

The automatic controlled rotation of the brush has a number of advantages:
Prevents joint diseases typical to hairdressers;
Stable and repeated rotation regardless of the hairdresser's state of alertness;
The preset rotation prevents uncontrolled automatic rotation that may cause entanglement of the brush in the hair;
Precise rolling for detaching the handle and leaving the roll in the hair.

When the brush is not turning, the handle 820 may be detached from the brush body 810, leaving the hair wound around the brush body 810 so that the brush body functions as a roll in the client's hair and may be fastened thereto by a rubber band 815 attached to the brush body 810.

The devices according to the present invention may be easily installed in conventional round hair brushes. Alternatively, hair brushes including the devices may be manufactured with different lighting and power generating capabilities, as described in the embodiments above.

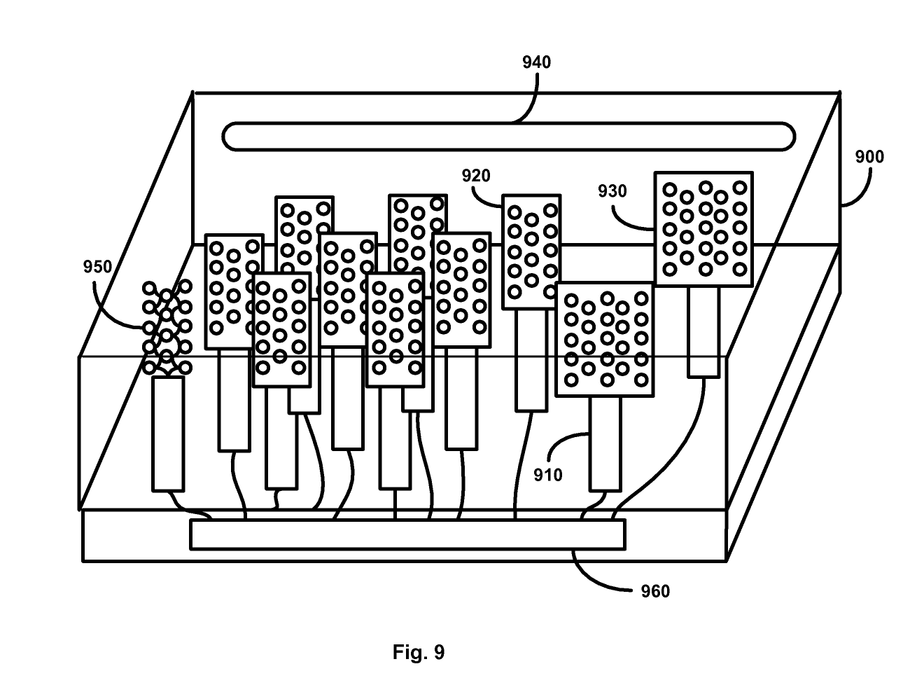

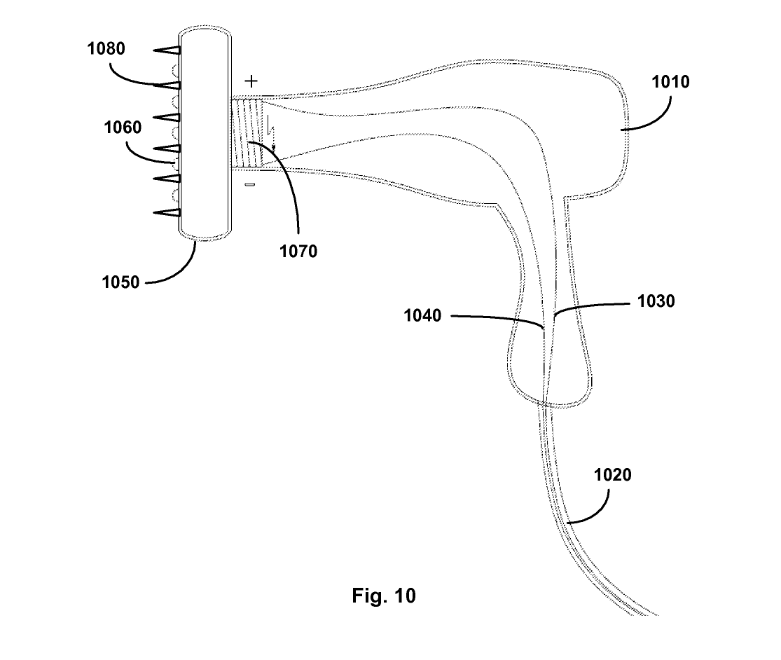

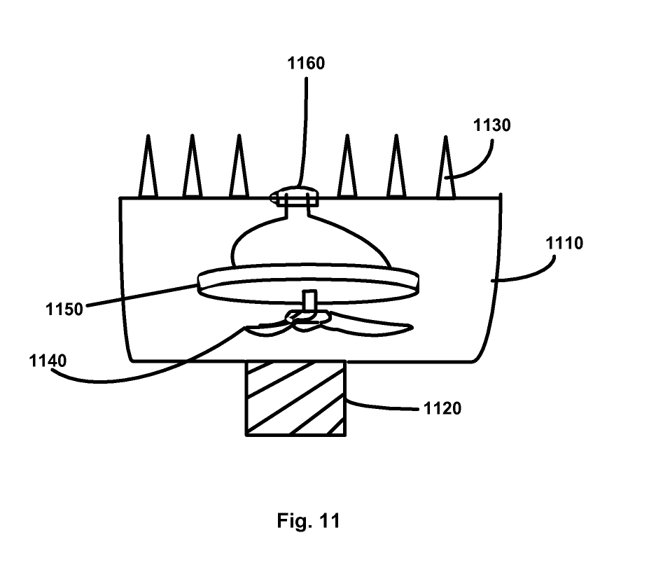

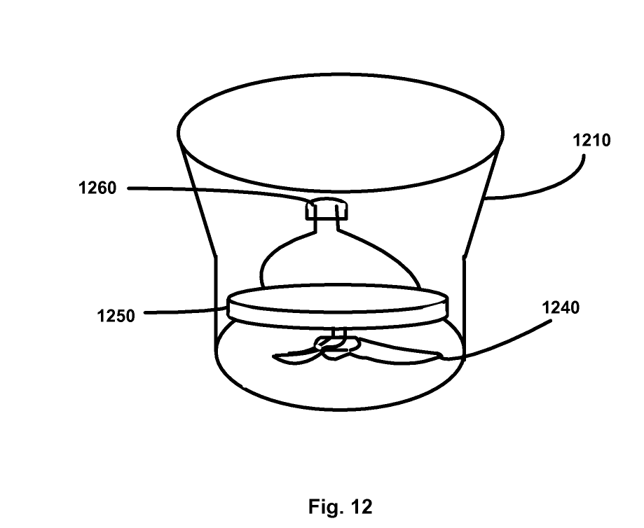

FIG. 9 is a schematic drawing of another embodiment of the present invention. According to this embodiment, hairbrush sterilization by UV light is done when the brush is not in use. The hairbrush, similar to the hairbrush depicted in FIG. 8, comprises UV LEDs and is detachable from its handle. The detached rolls may be accommodated in an especially designed receptacle 900, functioning as an internal sterilizer/sanitizer. Receptacle 900 comprises a plurality of carriers 910 connected to a power source 960, internal (as shown) or external, such as a battery or an electric socket. When a roll 920 is mounted on a carrier 910, the UV LEDs inside the roll are lit and sterilization of the roll takes place.

According to embodiments of the invention the brush includes both UV and IR LEDs, where during use of the brush only the IR LEDs are operated, and during sterilization in the sterilizer only the UV LEDs are operated.

In an alternative embodiment, roll 920 does not include UV LEDs, rather the UV LEDs 950 are mounted on the carrier and sterilize the roll/brush from within when it is inserted therein.

Internal sterilization of the roll/brush is important since it provides sterilization to areas otherwise difficult to reach with cleaning means, which may encourage growth of bacterial colonies.

Carriers 910 may be installed at varying distances, to accommodate rolls of different diameters, such as 920 and 930.

Additional UV lamps 940 may be installed inside the hairbrush sterilizer to provide external sterilization of the roll/brush.

The hairbrush sterilizer receptacle 900 may additionally comprise separate carriers serving as chargers for the battery (710, 855).

FIG. 10 is a schematic drawing of another embodiment of the present invention. According to this embodiment, a hair diffuser 1050 having protrusions 1080 as known in the art, additionally comprises at least one LED 1060 and is screwed 1070 into the front opening of a hair drier 1010 body. Hair dryer 1010 is connected to an electricity source (not shown) by an electric cable 1020 as known in the art. According to the embodiment of FIG. 10, electric cable 1020 comprises two additional heat resistant electric wires (1030, 1040), which pass electric power through the screwing 1070 to the one or more LEDs 1060. Electric wires (1030, 1040) may be connected at their distal end to the operating button of the hair drier, or to a different operating button for turning the LEDs on.

Structural and operational details of the embodiment of FIG. 10 are similar to the same details described in conjunction with the structure and operation of the embodiment of FIG. 7.

According to embodiments of the invention, additional pairs of heat resistant electric wires may be provided in electric cable 1020, where each pair may power LEDs of different wave length (e.g. UV, IR) mounted in the hair diffuser. The different LEDs may be operated together, using a single operating button, or a separate operating means may be provided for each griup of LEDs, e.g. a separate operating button or a digital operating panel.

FIG. 11 is a schematic drawing of another embodiment of the present invention. According to this embodiment, a hair diffuser 1110 having protrusions 1130 as known in the art, additionally comprises at least one LED 1160 and is screwed 1120 into the front opening of a hair drier (not shown). Hair diffuser 1110 additionally comprises an electric generator 1150, such as a dynamo generator and a propeller 1140 rotatably mounted on a shaft, which is in turn connected to the generator 1150 shaft (not shown).

Structural and operational details of the embodiment of FIG. 11 are similar to the same details described in conjunction with the structure and operation of the embodiment of FIGS. 2 and 3.

FIG. 12 is a schematic drawing of yet another embodiment of the present invention. According to this embodiment, an air concentrator 1210 configured to be attached to a hair drier, as known in the art, comprises at least one LED 1260. Air concentrator 1210 additionally comprises an electric generator 1250, such as a dynamo generator and a propeller 1240 rotatably mounted on a shaft, which is in turn connected to the generator 1250 shaft (not shown).

Structural and operational details of the embodiment of FIG. 12 are similar to the same details described in conjunction with the structure and operation of the embodiment of FIGS. 2 and 3.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof which would occur to persons skilled in the art upon reading the foregoing description.

1. A hair styling device comprising:
a hollow body comprising holes, said body comprising:
at least one electric generator having a shaft;
at least one LED electrically connected with said at least one electric generator; and
at least one propeller electrically connected with said at least one electric generator shaft, said at least one propeller configured to turn upon receiving air current from a hair-dryer and transfer the rotation power to the generator,
said generator configured to transform said rotation power to electric power thereby lighting up the at least one LED.

2. The hair styling device of claim 1, wherein said hollow body comprises a round hair-brush body connected to a handle.

3. The hair styling device of claim 1, wherein said hollow body comprises a hair diffuser configured to be attached to a hair dryer.

4. The hair styling device of claim 1, wherein said hollow body comprises an air concentrator configured to be attached to a hair dryer.

5. The hair styling device of claim 1, wherein said electric generator is a dynamo generator.

6. The hair styling device of claim 1, wherein said at least one LED is one of a UV LED and an IR LED.

7. The hair styling device of claim 1, wherein said at least one LED comprises a plurality of electrically chained LEDs.

8. The hair styling device of claim 7, wherein said plurality of LEDs are selected from the group consisting of: UV LEDS, IR LEDS and a combination thereof.

9. The hair styling device of claim 2, wherein said at least one electric generator comprises one electric generator electrically connected to at least one propeller.

10. The hair-brush of claim 9, wherein said electric generator is attached to the end of the hollow body proximate the handle.

11. The hair styling device of claim 9, wherein said electric generator is attached essentially in the middle of the hollow body.

12. The hair styling device of claim 11, wherein said electric generator is electrically attached to two propellers.

13. The hair-brush of claim 2, wherein said at least one electric generator comprises two electric generators electrically connected to two propellers respectively.

14. A round hair-brush comprising:
a hollow body comprising holes in its circumference; and
a handle connected with the body, said handle comprising a battery and an operation switch;
wherein said body comprises:
at least one LED electrically connected with said battery,
said battery configured to provide electric power to said at least one LED.

15. The hair-brush of claim 14, wherein said battery is rechargeable.

16. The hair-brush of claim 14, wherein said at least one LED is one of a UV LED and an IR LED.

17. The hair-brush of claim 14, wherein said at least one LED comprises a plurality of electrically chained LEDs.

18. The hair-brush of claim 17, wherein said plurality of LEDs are selected from the group consisting of: UV LEDS, IR LEDS and a combination thereof.

19. A round hair-brush comprising:
a hollow body comprising holes in its circumference; and
a handle detachably connected with the body, said handle comprising a battery and a motor electrically connected with the battery,
said motor mechanically connected with a central shaft of said hollow body,
said body comprising at least one LED electrically connected with said motor,
said central shaft additionally connected with a planetary gear,
said handle additionally comprising a first on/off switch configured to light up the at least one LED and a second on/off switch configured to control said motor operation, wherein the motor is configured to perform a predefined rotation amount.

20. The hair-brush of claim 19, wherein said battery is rechargeable.

21. The hair-brush of claim 19, wherein said at least one LED is one of a UV LED and an IR LED.

22. The hair-brush of claim 19, wherein said at least one LED comprises a plurality of electrically chained LEDs.

23. The hair-brush of claim 22, wherein said plurality of LEDs are selected from the group consisting of: UV LEDS, IR LEDS and a combination thereof.

24. The hair-brush of claim 19, additionally comprising a rubber band 815 attached to the brush body.

25. The hair-brush of claim 19, wherein said predefined rotation amount comprises 180 degrees.

26. The hair-brush of claim 19, additionally comprising a direction selection switch configured to select the rotation direction of the brush body.

27. A hair-brush sterilization device for sterilizing hollow round hair-brushes comprising a hollow body detachable from its handle, comprising:
a plurality of carriers, each configured to be inserted into a hollow brush body for internal sterilization; and
a power source connected with said plurality of carriers,
wherein each one of said hollow brush bodies comprise at least one LED, said power source configured to light said at least one LED.

28. The hair-brush sterilization device of claim 27, wherein said carriers are configured to accommodate hollow brush bodies of different sizes.

29. The hair-brush sterilization device of claim 27, wherein at least one of said carriers is electrically connected with at least one UV LED, said at least one UV LED configured to be inserted into said hollow body.

30. The hair-brush sterilization device of claim 27, additionally comprising a UV lamp configured to sterilize said hollow brush bodies externally.
